(19) United States
(12) Patent Application Publication
(10) Pub. No.: US 2011/0184283 A1
(43) Pub. Date: Jul. 28, 2011

(54) SYSTEM AND METHOD OF CLINICAL TREATMENT PLANNING OF COMPLEX, MONTE CARLO-BASED BRACHYTHERAPY DOSE DISTRIBUTIONS

(75) Inventor: Mark J. Rivard, Hopkinton, MA (US)

(73) Assignee: TUFTS MEDICAL CENTER, Boston, MA (US)

(21) Appl. No.: 13/055,850

(22) PCT Filed: Jul. 23, 2009
(86) PCT No.: PCT/US09/51555

§ 371 Date: Apr. 15, 2011

Related U.S. Application Data
(60) Provisional application No. 61/083,831, filed on Jul. 25, 2008.

(51) Int. Cl. A61B 6/00

(52) U.S. Cl. 600/436

(57) ABSTRACT

A system, method, and computer program product of clinical treatment planning implements complex Monte Carlo (MC) based brachytherapy dose distributions using conventional brachytherapy treatment planning systems (TPS). Dose distributions from complex brachytherapy source configurations determined with MC methods are used as inputs. Radial dose functions and 2D anisotropy functions are obtained by positioning the coordinate system origin along the dose distribution cylindrical axis of symmetry. Origin to tissue distance and active length are chosen to minimize TPS interpolation errors. A 2D anisotropy function is determined, and a brachytherapy dose rate constant is selected. A virtual brachytherapy source dose distribution is calculated based upon the complex treatment configuration. Additional dosimetry parameters may be considered as well, and dose distributions may be calculated and compared to the original MC-derived dose distributions. The present techniques may calculate dose to a specific tissue type instead of dose to water as used in the TG-43 formalism

### CROSS REFERENCE TO RELATED DOCUMENTS

The present application claims the benefit of priority of U.S. Provisional Patent Application Ser. No. 61/083,831 filed on Jul. 25, 2008. The contents of the U.S. Provisional Patent Application are incorporated below by reference.

### FIELD OF THE INVENTION

The present invention relates to systems and methods for utilization in brachytherapy in the fields of medical physics and therapeutic radiology. More specifically, the present invention relates to clinical treatment planning of complex, Monte Carlo-based brachytherapy dosimetric distribution protocols.

### BACKGROUND OF THE INVENTION

Radiation therapy refers to the medical treatment of diseases with ionizing radiation. Radiation therapy is often used in the treatment of neoplastic disease, especially solid, malignant tumors. In radiation therapy, the goal is to destroy the malignant tissue while at the same time minimizing radiation damage to other tissue, such as nearby healthy tissue, and minimizing the exposure of medical personnel to radiation. The recognized method employed for radiation treatment in body cavities such as the throat, bowel or vaginal region, and in regions of the body opened surgically or interstitially, is brachytherapy. Brachytherapy techniques place one or more radioactive sources inside or adjacent to an anatomical treatment site. The radioactive source placement may be precisely controlled and metered to the treatment site by an afterloading device. The radiation source is then moved to the treatment site to subject the tissue to radiation with a characteristic previously-calculated isodose contour. See, for example, Nath, et al., “Dosimetry of Interstitial Brachytherapy Sources: Recommendations of the AAPM Radiation Therapy Committee Task Group,” No. 43, Med. Phys. 22: 209-234 (1995); see also Lukas, et al., “Intraoperative Radiotherapy with High Dose Afterloading (Flabs Method) in Intraoperative Radiation Therapy,” Proceedings 4th International Symposium IORT, Schildberg and Kramling, eds., 1992 (Verlag Die Blaue Eule, Essen).

In conventional brachytherapy, the distance between the radioactive source and the tissue to be treated is relatively short (e.g., 0.1-5.0 cm). However, brachytherapy is a comprehensive term, and includes radiotherapy effected by interstitial, intercavitary, and surface application (plaque). Interstitial and intracavitary techniques are often advantageous where deep-seated lesions are involved, while plaque therapy is often advantageous where superficial or accessible diseased tissue is involved. In contrast, another form of radiation therapy called “external beam therapy” involves treatment at relatively large distances (i.e., 50-500 cm) between the radiation source and the skin surface. Accordingly, with “external beam therapy,” it generally is difficult to radiate the underlying diseased tissue yet spare the normal tissues that may also be in the path of the radiation beam directed towards the target site.

There are two general types of brachytherapy, those involving permanent implants and those which utilize temporary implants. Although a wide variety of radioactive elements (“radioisotopes”) have been previously proposed for therapeutic use, only a relatively small number have actually been accepted and employed on a large-scale basis. This is due, at least in-part, to a relatively large number of constraining considerations where medical treatment is involved. These considerations include the energy of the emitted radioactivity, the half-life of the element, the availability of the radioactive source, and the like. Radium is one radioactive element employed on a large-scale basis. Radium possesses a long half-life (i.e., approximately 1600 years) but also requires careful attention to the protection of medical personnel, as well as protection of the adjacent healthy tissue of the patient. This is due to radium's complex and highly penetrating gamma ray emission. To minimize exposure from the radioactive sources to medical personnel, specialized and sometimes complicated afterloading techniques have been developed whereby the radioisotope is guided, for example through a hollow tube or plastic catheter, to the treatment region following preliminary placement of the specialized guide appliances. The afterloading devices may be surgically implanted to allow placement of radioactive wires or seed ribbons with an afterloading procedure. Using conventional x-ray and dummy sources, imaging for implant localization may completely eliminate operating room staff exposure.

In addition to radium, other radioactive sources have been employed. For example, permanent implants may use radioactive particles, or “seeds,” containing iodine-125. Similarly, for temporary implants, cesium-137, iridium-192, and palladium-103 sources have been employed.

Historically, brachytherapy prescriptions were stated in terms of exposure and exposure rate. In intracavitary gynecologic brachytherapy, the use of mg h radium or radium-equivalent dosimetry was the standard for decades. Interstitial brachytherapy was often performed using rules specifying the geometric arrangement of radium needles and their radioactive contents relative to the target volume. The treatment time calculation was based on the implanted area or volume which was used to obtain exposure or mg h estimates from dose-specification criteria for idealized needle arrangements described by the system.

Based upon the shape of the brachytherapy implant, radioactive sources emit radiation in a measurable pattern. That is, the incidence and dosage of the radiation may be predicted, calculated, and measured based upon the shape and identity of the radioactive source. Using these calculations, an isodose map may be created to illustrate lines of equal absorbed radiation doses. In order to avoid harming the patient's healthy tissue and to ensure an accurate radiation dose is delivered, the radioactive source(s) must be accurately positioned and fixed on or in the patient. When the sources are accurately placed, the required isodose contours may be programmed. If the radiation source is not accurately positioned, there may be considerable overdosage to normal (i.e., non-tumorogenic) tissue, with serious risk of harm to the patient, or exposure of medical staff to radiation. Additionally, in cases of repeated radiation treatments, where a reduced radiation dose is given in each subsequent treatment, accurate localization of the radioactive source(s) at the site of treatment over a lengthy period is of particular importance.

Classically, the dose rate for a source is derived from its contents, that is the amount of radioactivity contained within the source. The activity was first expressed as the weight of the contents, for ²²⁶Ra in mg. The unit “Ci” is defined as the activity of 1 g of ²²⁶Ra. The dose rate in Gy/h at distance r from a point source is described as:

$${\dot{D}}_{W} ( r ) = \frac{A \cdot Γ \cdot {f}_{as , W} ( r ) \cdot {F}_{m}}{{r}^{2}} , \qquad ( 1 )$$

in which A is the effective activity in Ci, deduced from dose rate measurements in air, Γ is the exposure rate per Ci of the radionuclide in R/h at 1 m, f_{as,W}(r) is the correction for absorption and scatter effects of the photons at distance r in water when compared to the same point in vacuo, and Fₘ is the medium conversion factor from R to Gy.

Inspection of this equation reveals a number of flaws and possibilities for errors. First, there is the confusion between the real and the effective, assumed, or apparent radiation activity. Second, at least quantitatively, a transmission medium is poorly defined in relation to the value of the Fₘ factor. It differs for water, tissue, and materials with strongly deviating composition, such as bone and lung. These values must be taken from textbooks or other resources where there are large (˜25%) disparities in the tabulated values. This is apparent when trying, for example, to list Γ values from the literature for a radionuclide with a complex photon spectrum such as ¹⁹²Ir. These flaws may lead to dosimetry errors, for example, when the effective activity is measured from dose rate in air by the radionuclide vendor while the user of a given calculation system has included a different Γ value.

The quantities for A, Γ, and Fₘ in Eq. (1) are not in accordance with the International System of Units (SI system), and are therefore considered obsolete. Eq. (1) may to be rewritten for the dose rate to water D_{W}(r). The rewritten calculations result in Eqs. (2) and (3),

$$\begin{matrix}{\dot{D}}_{W} ( r ) = {\dot{K}}_{R} ( 1 - {g}_{α} ) {( \frac{{μ}_{en}}{ρ} )}_{α}^{W} {( \frac{{r}_{0}}{r} )}_{{f}_{as , W}}^{2} ( r ) , & ( 2 ) \\ {\dot{D}}_{W} ( r ) = {S}_{K} ( 1 - {g}_{α} ) {( \frac{{μ}_{en}}{ρ} )}_{α}^{W} {( \frac{1}{r} )}_{{f}_{as , W}}^{2} ( r ) . & ( 3 )\end{matrix}$$

The reference air-kerma rate \dot{K}_{R} and air-kerma strength S_{K} are mutually related through the inverse-square law to the reference distance r₀ as \dot{K}_{R}=S_{K}(1/r₀)². gₐ is the fraction of electron energy liberated by photons in air that is lost due to radiative processes such as bremsstrahlung and fluorescence. The term (μₑₙ/ρ)_{α}^{W} is the ratio of the mass-energy absorption coefficient of water to that of air.

A real radiation source has finite dimensions compared to an idealized or theoretical point source. The most common shape of a brachytherapy source is the cylinder. The design of the source, its shape, and the choice of the active material influence the resulting dose distribution. Bremsstrahlung photons generated in the capsule by beta particles may contribute to the dose, and the capsule may filter the radiation emitted by the bare source material. The photon energy spectrum of a realistic source can therefore deviate substantially from that of a bare source, with especially significant deviations along the source long axis. The calculation process according to the Sievert-integration procedure considers the dose at a point near the source as the summation of point-source contributions. An elongated source is then divided into a series of infinitesimal source elements. The path-length attenuation through the source and the capsule wall using the effective-attenuation coefficients for the small source elements takes into account most of the effects. This method may be accurate to within 2%-8% for ¹³⁷Cs sources but is less accurate at low- and intermediate-photon energies (e.g., less than 0.4 MeV).

Previous dose calculation algorithms have been based on the use of tabulated data in Cartesian or polar coordinates, or have made use of directly calculated data from the Sievert-integration procedure. The conventional manual and the classical approaches in dose-calculation algorithms used in computerized systems are rather simplistic. Generally, the influence of tissue heterogeneity, organ or body outline, or applicator heterogeneity is ignored with the exception of an occasional one-dimensional (1D) correction to the total dose to account for a predefined ovoid shield. Interseed attenuation (ISA) or interapplicator shielding is not addressed. Other effects are difficult to quantify since they depend on many patient-specific circumstances.

The conventional approach for brachytherapy dose calculation is based on the AAPM TG-43 dosimetry formalism, which yields calculated dose rates for the radioactive elements and relies on superposition of single-source dose distributions obtained in a liquid water phantom with a fixed volume for radiation scattering. This approach was developed through computerized treatment planning systems (TPSs) to replace antiquated manual approaches.

Based on the assumption of cylindrically symmetric brachytherapy source dose distributions, the TG-43 brachytherapy dosimetry formalism utilizes a polar coordinate system along the source long axis (z axis) with the coordinate system origin located at the center of radioactivity, as shown in FIG. 2. Dose distributions at point P(r, θ) are obtained in the vicinity of the source with radial distance r and polar angle θ expressed relative to the origin and source long axis, respectively. A special reference point P (r₀, θ₀) is positioned at r₀=1 cm and θ₀=90° on the transverse plane of the source. For FIG. 2, β=θ₂−θ₁, and t is the capsule thickness in the transverse-plane direction.

Conventional TPS may use either the 1D or 2D equation to calculate dose rate as shown in Eqs. (4) and (5), respectively, with dosimetry parameters defined below,

$$\begin{matrix}D ( r ) = {S}_{K} \cdot Λ \cdot {( \frac{{r}_{0}}{r} )}^{2} \cdot {g}_{p} ( r ) \cdot {φ}_{an} ( r ) , & ( 5 ) \\ D ( r , θ ) = {S}_{K} \cdot Λ \cdot \frac{{G}_{L} ( r , θ )}{{G}_{L} ( {r}_{0} , {θ}_{0} )} \cdot {g}_{L} ( r ) \cdot F ( r , θ ) . & ( 6 )\end{matrix}$$

Identical to Eqs. (2) and (3), S_{K} is the air-kerma strength of the brachytherapy source and is determined for specific sources either by a clinical medical physicist or by the manufacturer. S_{K} has units U=cGy cm² h⁻¹ and its measured value should be traceable to a primary standards dosimetry laboratory (PSDL) such as the National Institute of Standards and Technology (NIST). The dose rate constant Λ has units of cGy h⁻¹ U⁻¹ such that Λ=\dot{D}(r₀θ₀)/S_{K} since the other dosimetry parameters from Eq. (5) take values of unity at P(r₀, θ₀). Dose rate variations in the source transverse plane are accounted for by the geometry function and radial dose function. The geometry function takes the simple form of point-source or inverse-square approximation (r₀/r)² for the 1D formalism as is often used in brachytherapy TPS for LDR low-energy photon-emitting brachytherapy using sources such as ¹²⁵I. However, the 2D formalism approximates the distribution of radiation emissions as a line-segment with length L and is often used for HDR high-energy photon-emitting brachytherapy sources such as ²⁹²Ir. Here, G_{L}(r, θ)=β/L·r sin(θ). The point-source radial dose function is given by g_{P}(r)=\dot{D}(r)/\dot{D}(r₀)·(r/r₀)², whereas the line-source radial dose function is g_{L}(r)=\dot{D}(r,θ₀)/\dot{D}(r₀,θ₀)·G_{L}(r₀,θ₀)/G_{L}(r,θ₀). For calculating dose rate off the transverse-plane where dose anisotropy from brachytherapy sources comes into play, the 1D anisotropy function φₐₙ(r) or the 2D anisotropy function F(r, θ) may be used.

Often there are several dosimetry publications on a given brachytherapy source model, and thus there is potential for variable administration of dose with clinically relevant ramifications. To subjugate this problem, the AAPM prepares consensus dosimetry datasets for use in brachytherapy TPS. Multiple consensus datasets for LDR ¹²⁵I and ¹⁴³Pd sources have been issued by industry standard reports such as the AAPM TG-43U1 and TG-43U1S1 reports. These datasets are incorporated by TPS software vendors as machine data, and are also used in practice for treatment planning of patients. Incorrect application of the dosimetry data sets could cause variability in clinical practice and unnecessary variations in administered dose or even serious dose-calculation errors.

While dataset standardization is a key element for acquisition of high-quality clinical results, dosimetry parameter datasets used in the AAPM TG-43 dosimetry formalism are obtained in a liquid water phantom with a fixed volume for radiation scattering. Consequently, the conventional formalism does not readily account for several important aspects that undermine clinical application of the TG-43 formalism with these datasets.

A basic understanding of radiological physics interactions can generally explain many of the limitations of the AAPM TG-43 dosimetry formalism. Under charged-particle equilibrium, the mass-energy absorption coefficient as a function of photon energy E and atomic number Z, noted as (μₑₙ/ρ)_{E,Z}, may be used to account for differences in absorbed dose between water and tissue as Dₜᵢₛₛᵤₑ(μₑₙ/ρ)_{E,tissue}=D_{water}(μₑₙ/ρ)_{E,water}. Using the soft tissue characterization from the International Commission on Radiation Units (ICRU 44), with water and tissue mass densities of 1.00 and 1.06 g/cm³, FIG. 3 presents the effect of phantom medium on absorbed dose and attenuation as the ratio of absorbed dose in water to tissue Dₜᵢₛₛᵤₑ^{water} as a function of photon energy. Over the range of photon energies examined, 0.01-10 MeV, the absorbed dose in tissue is nearly equal to that in liquid water as shown by the solid curve. This ratio dips to 0.96 near 0.05 MeV due to slightly higher photoelectric effect cross sections of tissue compared to water.

Similarly, the mass-attenuation coefficient as a function of photon energy and atomic number (μ/ρ)_{E,Z} may approximate differences in radiation attenuation between water and tissue I=I₀e^{-ρZ·(μ/ρ)E,Z}^{^{−1}} or I=I₀e^{-μE,Z}^{^{−1}}. Using the water and tissue mass densities, but with a simple path length approach using t=1 cm, the ratio of radiation attenuation between the two media (I/I₀)_{water}/(I/I₀)ₜᵢₛₛᵤₑ=e^{-μ}^{_{E,water}}^{-1 cm}/e^{-μ}^{_{E,tissue}}^{-1 cm} is presented in FIG. 3 as the dashed curve 310. Unlike the absorbed dose ratios, the larger mass density of tissue contributes to the increased attenuation. Further, increased attenuation by tissue below 0.05 MeV is caused by the larger photoelectric effect cross section of tissue compared to water.

For multisource clinical applications, interseed attenuation (ISA) may be significant. Applicator to radiation interactions and applicator shielding may detract from the accuracy of conventional TG-43 based dose calculations. For radiation shielding between sources or by an applicator, the attenuation of water is replaced with that of a high-Z material (that is, an element with a large number of protons). For LDR low-energy photon-emitting sources, this high-Z material may, for example, be a silver (Z=47) radio-opaque marker used for identification during postimplant CT localization. For HDR high-energy photon-emitting sources, the material may be a stainless steel applicator (Z=26). Using ρ_{Ag}=10.5 g/cm³ and ρ_{Fe}=7.8 g/cm³ with a minimal thicknesses of 0.1 mm and negligible water attenuation, the high-Z photon attenuations are given in FIG. 4 where photon transmission ratios 410, 420 through water or high-Z attenuators, respectively, are shown as functions of photon energy. As in the prior two examples, the photoelectric effect is largely responsible for the differences in comparison to water.

Phantom dimensions and volume affect Monte Carlo (MC) dosimetry calculations. Monte Carlo dosimetry calculations are based upon random sampling of particle histories to precisely estimate the absorbed dose. FIG. 5 shows a comparison of ¹⁹²Ir g(r) values calculated for a given spherical water phantom radius R compared to a base radius of R=50 cm, which is assumed to provide full scatter conditions. Data include curves 505, 510, 515, 520, 525, 530, 535, 540, 545 for R=5, 10, 15, 20, 25, 30, 35, 40, and 45 cm, respectively. For high-energy photon-emitting brachytherapy sources such as ¹⁹²Ir, dose differences greater than 5% are possible within 10 cm of the source, as shown in FIG. 5. However, the different phantom dimensions quantifies only the effect of excess material or missing backscatter close to the phantom boundary as source position is fixed and phantom radius decreases. However, in actual clinical practice, where a source dwell position is programmed close to the patient contour as scattering conditions are altered in total, including lateral scatter. Further, the proportion of dose due to primary and scattered radiation significantly changes as a function of distance from the source.

Subtleties associated with dose calculation include the assumption of equivalence of absorbed dose and kerma. Kerma is the kinetic energy released per unit mass. The absorbed dose is the energy absorbed in the mass. Charged-particle disequilibrium in homogeneous media due to brachytherapy inverse-square may increase in photon fluence upon moving toward a brachytherapy source. Concern for dose to kerma equivalence is not commonly observed in brachytherapy measurements or MC calculations of conventional high-energy sources since the required distance is ˜5 mm for greater than a 5% effect. For LDR ¹³⁷Cs sources, this distance is adjacent to the source capsule. For HDR ¹⁹²Ir or ⁶⁰Co sources, dose/kerma of ≧1.05 occurs at distances below 2 and 7 mm from the source center, respectively. Breakdown of charged particle equilibrium (CPE) can also occur at material boundaries such as tissue-to-high-Z interfaces. The dose contribution from betas emitted upon radionuclide disintegration is another aspect of electrons in brachytherapy dosimetry ignored with conventional TPS.

Absorbed dose in water is about −4% and +2% compared to tissue for low- and high-energy photons, respectively. Per centimeter, the attenuation of high-energy photons is about the same between water and tissue. However, there are significant differences in attenuation for low-energy photons, and the differences increase as photon energy decreases. The presence of high-Z materials also can substantially alter dose distributions for low-energy photons. Dose differences of >5% are possible for high-energy sources within 5 cm of the skin. Equivalence of dose and kerma within 5% does not hold true within a few millimeters of high-energy photon-emitting sources. Dosimetric contributions from beta emissions at these distances are also ignored in the current TG-43 formalism.

In addition to dosimetric limitations, there are limitations to applying the AAPM TG-43 dosimetry formalism to certain clinical circumstances. For instance, the formalism does not readily permit calculation of dose distributions for curved brachytherapy sources such as LDR ¹⁹²Ir wire. This is also true for long sources where \dot{D}(r<L/2,θ₀) is needed, but is difficult to implement on TPS with the 2D formalism since high F(r, θ) gradients near the source long axis require high-resolution dosimetry parameters. Beyond these TPS limitations, the scope of anatomic sites commonly considered for application of brachytherapy are practically subject to the limitations of the AAPM TG-43 dosimetry formalism outlined above. A breakdown is presented in Table 1 of the relative sensitivity of these anatomic sites to the five dosimetric limitations. The sensitivity of commonly treated anatomic sites to dosimetric limitations of the current brachytherapy dose calculation formalism is shown. Items flagged as “Y” indicate significant differences between administered and delivered dose are possible due to the highlighted dosimetric limitation.

TABLE 1

| | Source | Absorbed | | | | Beta/kerma |
|---|---|---|---|---|---|---|
| Anatomic site | energy | dose | Attenuation | Shielding | Scattering | dose |
| Prostate | High | N | N | N | N | N |
| | Low | Y | Y | Y | N | N |
| Breast | High | N | N | N | Y | N |
| | Low | Y | Y | Y | N | N |
| GYN | High | N | N | Y | N | N |
| | Low | Y | Y | N | N | N |
| Skin | High | N | N | Y | Y | N |
| | Low | Y | N | Y | Y | N |
| Lung | High | N | N | N | Y | Y |
| | Low | Y | Y | N | Y | N |
| Penis | High | N | N | N | Y | N |
| | Low | Y | N | N | Y | N |
| Eye | High | N | N | Y | Y | Y |
| | Low | Y | Y | Y | Y | N |

Table 1 highlights the sensitivity of each anatomic site to the aforementioned dosimetric limitations. Prostate implants using high-energy sources such as HDR ¹⁹²Ir are deep seated and are not as sensitive to radiation scattering conditions as are other sites. Further, a single source is translated throughout the implanted plastic catheters, and thus none of the other effects are as prominent. For low-energy photon-emitting sources in the prostate, the D₉₀ is generally within 1 cm of the gland, and water to tissue attenuation differences are minimal. However, absorbed dose can differ by several percent, and ISA can exceed 10% along the needle direction. LDR prostate implants typically utilize the largest number of implanted sources among all anatomic sites, and may be subject to dosimetric effects of calcifications. Breast implants using HDR ¹⁹²Ir share similar circumstances as HDR ¹⁹²Ir prostate implants. However, the radiation scattering conditions differ and generally overestimate dose deposition in clinical implants, especially near the skin surface. For breast implants using low-energy photon emitting sources such as LDR ¹⁰³Pd or a 50 kVp source, scattering differences are negligible between the treatment plan using superposition of source locations in liquid water and the clinical environment. However, absorbed dose in breast tissue at these energies is generally underestimated by several percent, source-to-source shielding can be significant for ¹⁰³Pd sources, and differences between prescribed and administered doses can also be significant due to differences in radiation attenuation between water and tissue, especially at large distances. Gynecological implants often use high-Z colpostats for shielding the bladder and rectum, particularly when treating the uterus. For treating the vagina/cuff, treatment through a plastic applicator is de rigueur and contributes to differences in radiation attenuation. Absorbed dose in these situations is underestimated by several percent for low-energy sources. However, radiation scattering effects do not need to be considered due to the deep-seated position within the body. Brachytherapy of the skin is most sensitive to scattering conditions. Use of bolus material provides better agreement between planned dose and delivered dose, but skin dose outside the treatment field can be minimized without bolus. Further, most high- and low-energy brachytherapy treatments of the skin use some form of shielding to protect the unintended tissues, and conventional TPS cannot account for this shielding. Again, absorbed dose is underestimated by several percent for low-energy brachytherapy sources. Lung implants typically include tissues with mass densities three to four times less than conventional tissues. Consequently, scattering conditions for both high- and low-energy brachytherapy sources are not congruent between the clinical implant and the planned approach. Absorbed dose and attenuation are also significantly different for low-energy sources, and dose calculations close to high-energy sources such as HDR ¹⁹²Ir can be in error by several percent within a few millimeters of the source. Brachytherapy of the penis is performed using either HDR high-energy sources or LDR low-energy sources. Both applications overestimate administered dose due to radiation scattering conditions. Further, low-energy sources underestimate dose due to differences in tissue composition between water and tissue. Eye plaque brachytherapy has been conducted for several decades but is sensitive to all of the aforementioned dosimetric limitations.

In summary, it is clear that most applications of brachytherapy are subject to limitations of the current dose-calculation formalism. Thus, it is imperative that advances be made to resolve these discrepancies and provide clinical users a realistic depiction of individualized brachytherapy dose administration.

### SUMMARY OF THE INVENTION

Brachytherapy is a mature treatment modality that has benefited from technological advances. Treatment planning has advanced from simple lookup tables to complex, computer-based dose-calculation algorithms. The present invention includes a system, method, and computer program product for clinical treatment planning of complex, Monte Carlo-based brachytherapy dose distributions.

As indicated above, conventional brachytherapy approaches are based on the AAPM TG-43 formalism. However, this formalism has clinically relevant limitations for calculating patient dose. While certain brachytherapy dose distributions, such as those for LDR prostate implants, are readily modeled by treatment planning systems (TPS) that use the superposition principle of individual seed dose distributions to calculate the total dose distribution, dose distributions for brachytherapy treatments using high-Z shields or having significant material heterogeneities are not currently well modeled using conventional TPS.

The present invention includes a system, method, and computer program product for clinical treatment planning of complex Monte Carlo-based brachytherapy dose distributions without the shortcomings of previous techniques. A system, method, and computer program product in accordance with the present invention establishes a new treatment planning technique that may be applied in clinical situations where conventional approaches are not acceptable and dose distributions present cylindrical symmetry.

In a system and method in accordance with the present invention, dose distributions from complex brachytherapy source configurations determined with Monte Carlo methods are used as input data. Radial dose functions and 2D anisotropy functions are obtained by positioning the coordinate system origin along the dose distribution cylindrical axis of symmetry. Origin-to-tissue distance and active length may be chosen to minimize TPS interpolation errors. Dosimetry parameters are entered into the TPS, and dose distributions are subsequently calculated and compared to the original Monte Carlo-derived dose distributions. The dosimetry planning technique of the present invention may reproduce brachytherapy dose distributions for many applicator types, producing dosimetric agreement typically within 2% when compared with Monte Carlo-derived dose distributions. Agreement between Monte Carlo-derived and planned dose distributions improves as the spatial resolution of the fitted dosimetry parameters improve. The system and method of the present invention incorporates complex Monte Carlo-based brachytherapy dose distributions into conventional therapy planning systems and provides dosimetry calculation results that are generalizable to other brachytherapy source types and other therapy planning systems.

Monte Carlo (MC) techniques use stochastic approaches (random numbers) to sample probability density functions describing the phenomena underlying the transport of particles through matter for simulations. With sufficient statistics or particle histories, MC obtains precise solutions to a variety of problems in radiation therapy. Another calculation approach consists of describing the statistical particle distribution by directly solving the linear Boltzmann transport equation (LBTE) through deterministic means. Two general approaches with application to brachytherapy include solving the differential LBTE and the integral formulation. The LBTE may be solved by discretization of the parameters phase space. One method is to focus on the angular domain or the use of DO. Discretization is also performed in space (finite difference or finite element) and in energy with appropriate multigroup cross sections. The system can be coupled to handle neutral, charged, and coupled photonelectron-positron transport for iterative solutions. The discretization of the space dimension may be well suited to problems in radiation therapy where voxel-based geometries of patients are constructed from tomographic images.

The accuracy of deterministic approaches is directly related to discretization, or transferring continuous models and equations into their discrete components. Fine discretization steps lead to accurate solutions at the price of a larger system of equations to be solved. Thus, the technique is numerically intensive. The deterministic concept of accuracy is not the same as the concept of accuracy for MC dose calculation and is related to a systematic difference between the solution and the “truth.” With deterministic tools, uncertainty does not have a stochastic component, while MC uncertainties have stochastic and systematic components. An important issue for brachytherapy is the “ray effect,” which results from the nonphysical fluence buildup due to the limited number of angles for near pointlike sources in low-density media. To diminish this artifact, stochastic or semi-analytic methods may be used to determine the once-scattered dose distribution within the discretized phase space.

Direct Monte Carlo (MC) simulation is based on a random sampling of particle histories to estimate the quantity of interest of absorbed dose in the patient. MC plays an important role in several aspects of brachytherapy dose calculation. For example, MC methods revealed theoretically that dose rates from ¹²⁵I were lower by 10%-14% due to air-kerma contributions from titanium characteristic x rays in the NIST S_{K,N85} calibration standard. MC is an integral part of the confirmatory process and is an equal to measurements for AAPM brachytherapy dosimetry parameter datasets. MC simulation is also used to characterize radiation sources in terms of their spatial distribution of primary and scattered radiation to allow simple input data for modeling clinical sources. Further, MC can be used to derive transmission data through materials and thus contributes to radiation protection data. MC methods are a key tool to characterize shielding effects, ISA, and other relevant factors for clinical brachytherapy dose calculation.

Monte Carlo methods are the most accurate method for predicting dose distributions. The path of the particles (electrons, photons, protons, or neutrons) through the patient are simulated by Monte Carlo software algorithm calculations. Particle interactions (scattering, attenuation, and energy deposition) are simulated one at a time. Monte Carlo methods trace paths of several million particles through a patient model, where the patient model accurately reflects three dimensional variations of electron density within the volume of the patient under study. For large numbers of source radiation particles (typically above 10⁷), the Monte Carlo method produces an accurate representation of the dose distribution. For these reasons, the Monte Carlo method is clinically preferred for the calculation of radiation dose in electron beam radiotherapy.

However, the MC calculation time for direct brachytherapy applications is affected by the inverse-square law fall off of primary photon fluence over distance, which leads to a low density of primary photons at a distance. Another problem is the nearly elastic scattering at relatively low energies, as it requires many interactions until the initial energy is lost. Such reasons have obstructed the use of MC algorithms in direct calculation of dose distributions around implants. Methods have been attempted to increase MC calculation speed by employing variance reduction techniques, such as correlated sampling. Additional calculation techniques may use dedicated source phase-space files to avoid tracking particles within brachytherapy seeds but allow full calculation of ISA, arbitrary seed orientation and material heterogeneities.

Others techniques have been used to pursue MC integration by generating voxel-based geometry from clinical implants exported directly from the TPS and imported into general-purpose codes. This has enabled retrospective dose comparison studies, essential when considering new dose-calculation methods.

Even though MC has been an integral part of many different aspects of brachytherapy, prior to the system and method of the present invention, commercial implementation into a brachytherapy TPS was unavailable.

A system and method in accordance with the present invention determines a virtual brachytherapy total dose distribution in a patient for radiation therapy treatment planning. The system and method includes receiving a Monte Carlo dose distribution from a brachytherapy source in a brachytherapy processing device. The system and method identifies a virtual brachytherapy source dose distribution with a cylindrical axis of symmetry. The system and method further includes determining an origin location of the virtual brachytherapy source and selecting an active length of a virtual brachytherapy source. A radial dose function is derived along a long axis of the virtual brachytherapy source dose distribution. The system and method then derives a 2D anisotropy function of the virtual brachytherapy source. The system and method selects a virtual brachytherapy dose rate constant at a radial distance reference point to reproduce the Monte Carlo dose distribution and calculates a virtual brachytherapy source dose distribution for a treatment configuration based upon the received Monte Carlo dose distribution.

The Monte Carlo dose distribution may be cylindrically symmetric, or it may be a complex configuration. Additionally, the system and method of the present invention may determine dosimetry parameters including patient scatter conditions, material heterogeneities, non-water radiation attenuation, and high-Z shielding.

Further, the system and method of the present invention may be used to correct for material heterogeneities between the patient and the virtual brachytherapy source, dose attenuation in collimated regions of the virtual brachytherapy source dose distribution, patient scatter conditions, and high-Z shielding.

The virtual brachytherapy source dose distribution of the present invention complies with the TG-43 dosimetry formalism. However, the radial distance reference point determined by the system and method of the present invention may not be equal to the AAPM TG-43 normalization reference point. The determined radial distance reference point may be chosen to minimize radial dose function interpolation errors.

A radial dose function may be derived in accordance with the present invention by positioning the origin location of the virtual brachytherapy source along the dose distribution cylindrical axis of symmetry. Further, the radial dose function may be derived to account for dose falloff and attenuation along the central (longitudinal) axis.

The system and method of the present invention may derive the 2D anisotropy function by positioning the origin location of the virtual brachytherapy source along the dose distribution cylindrical axis of symmetry. The 2D anisotropy function may be derived to minimize differences with the Monte Carlo dose distribution from the virtual brachytherapy source.

The active length of the virtual brachytherapy source utilized in the system and method of the present invention may define the virtual source as linear and include the 2D anisotropy function in calculating the virtual brachytherapy source dose distribution. For example, the linear virtual source length may be approximated as a point. Likewise, the virtual brachytherapy dose rate constant may reproduces the Monte Carlo dose rate distribution using the point-like linear virtual source and the derived 2D anisotropy function.

A radiation therapy planning system in accordance with the present invention is configured to determine a virtual brachytherapy total dose distribution in a patient for radiation therapy treatment planning. The system includes a brachytherapy processing device configured to receive a Monte Carlo dose distribution from a brachytherapy source. The brachytherapy processing device is further configured to identify a virtual brachytherapy source dose distribution with a cylindrical axis of symmetry, determine an origin location of the virtual brachytherapy source, and select an active length of a virtual brachytherapy source. Additionally, the brachytherapy processing device is also configured to derive a radial dose function along a long axis of the virtual brachytherapy source dose distribution, derive a 2D anisotropy function of the virtual brachytherapy source, and select a virtual brachytherapy dose rate constant at a radial distance reference point to reproduce the Monte Carlo dose distribution. The brachytherapy processing device is further configured to calculate a virtual brachytherapy source dose distribution for a treatment configuration based upon the received Monte Carlo dose distribution.

In addition, the system of the present invention includes a device for producing voxelized regions-of-interest for a patient to be treated. The voxelized regions-of-interest may be based on a plurality of clinical imaging data, such as computed tomography images, MRI images, and the like. The system further includes a data storage device and an image reconstruction device to produce and reconstruct patient or other clinical data to be used in a radiation therapy plan. The image reconstructing device may reconstruct cross-sectional images or other images from the voxelized regions-of-interest. Additionally, the system of the present invention includes an image-developing device for developing a translucent or other image of the patient. The developed image may be viewed from a predetermined point-of-view or may be adjusted by an operator using an operator entry input device. The system further includes a display device for displaying the received voxelized regions-of-interest and the reconstructed images produced by the radiation therapy planning system. The operator entry input device may be further configured for determining the isocenter location of a planned dose and a radiation angle over the received images. The operator entry input device may also be configured to display and adjust a radiation field over the translucent image displayed on the display device. Likewise, the operator entry input device may be further configured to adjust, modify, and change the isocenter location, the radiation angle, and the radiation field over the cross-sectional images displayed on the display device.

Systems and methods in accordance with the present invention extend the capabilites of brachytherapy beyond the limitations of the current dose-calculation formalism and provide advances that resolve the present formalism discrepancies and provide clinical users a realistic depiction of individualized brachytherapy dose administration.

These and other advantages, aspects, and features of the present invention will become more apparent from the following detailed description of embodiments and implementations of the present invention when viewed in conjunction with the accompanying drawings. The present invention is also capable of other embodiments and different embodiments, and details can be modified in various respects without departing from the spirit and scope of the present invention. Accordingly, the drawings and descriptions below are to be regarded as illustrative in nature, and not as restrictive.

### BRIEF DESCRIPTION OF THE DRAWINGS

The accompanying drawings illustrate an embodiment of the invention and depict the above-mentioned and other features of this invention and the manner of attaining them. In the drawings:

FIG. 1 illustrates system for clinical treatment planning of complex Monte Carlo-based brachytherapy dose distributions in accordance with the present invention.

FIG. 2 shows a coordinate system for the AAPM TG-43 brachytherapy source dosimetry formalism in accordance with the present invention.

FIG. 3 shows an effect of a phantom medium on an absorbed dose and attenuation.

FIG. 4 illustrates photon emission ratios through attenuators as a function of photon energy.

FIG. 5 shows a comparison of radial dose functions for a number of radii.

FIG. 6 illustrates a treatment planning coordinate system indicating an origin position for a virtual source in accordance with the present invention.

FIG. 7 shows a dose distribution comparison of Monte Carlo input and TPS output using a system and method in accordance with the present invention.

FIG. 8 illustrates a dose distribution comparison of a TPS output using a system and method in accordance with the present invention and a conventional planning system.

FIG. 9 illustrates a dose distribution comparison for a fully loaded 16 mm diameter eye plaque.

FIG. 10 shows a process flow chart for a method of clinical treatment planning of complex, Monte Carlo-based brachytherapy dose distributions in accordance with the present invention.

### DETAILED DESCRIPTION OF THE INVENTION

The following detailed description of the invention refers to the accompanying drawings and to certain preferred embodiments, but the detailed description does not limit the invention. The scope of the invention is defined by the appended claims and equivalents as it will be apparent to those of skill in the art that various features, variations, and modifications can be included or excluded based upon the requirements of a particular use.

The American Association of Physicist in Medicine (AAPM) Task Group No. 43 (TG-43) brachytherapy dosimetry formalism is well established and permits calculation of dose distributions for diverse circumstances, including low-dose rate (LDR) permanent prostate implants, gynecological implants using temporary high-dose rate (HDR) ¹⁹²Ir implants, and temporary episcleral plaque brachytherapy procedures. Conventional treatment planning systems (TPS) use the AAPM TG-43 brachytherapy dose calculation formalism, which is based on applying the source superposition principle to cylindrically symmetric single-source photon-emitting brachytherapy dose distributions throughout a clinically defined volume. Modern TPS software is capable of generating high-resolution dose distributions and dose volume histograms (DVHs) based on user-defined source positions and regions of interest. The conventional brachytherapy TPS algorithm provides acceptable treatment planning where source-to-source shielding is negligible, where water is radiologically equivalent to tissue over the appropriate photon energy range, where high-Z shields or low material densities (e.g., air) are not present, and when the scattering conditions for the clinical circumstances are similar to those present for acquisition of the initial, single-source brachytherapy dose distributions obtained using either measurements or Monte Carlo (MC) methods.

This conventional approach is acceptable for calculation of single-source dose distributions in unbounded water media. However, in clinical situations where complex brachytherapy sources or applicators with high-Z shielding or bounded-phantom configurations are present, the conventional brachytherapy TPS algorithms fail to provide an accurate calculation of clinical brachytherapy dose distributions. In fact, it is not possible in some instances to perform treatment planning using the conventional approach.

In the system and method of the present invention, dose distributions from complex brachytherapy source configurations determined with Monte Carlo methods are used as input data. These source distributions may include, for example, the 2 and 3 cm diameter skin applicators, 4-8 cm diameter peripheral breast brachytherapy applicators, and a 16 mm eye plague using ¹⁰³Pd, ¹²⁵I, and ¹³¹Cs seeds. Of course, other applicators may also be used.

Radial dose functions and 2D anisotropy functions are obtained by positioning the coordinate system origin along the dose distribution cylindrical axis of symmetry. Origin-to-tissue distance and active length may be chosen to minimize therapy planning system (TPS) interpolation errors. Dosimetry parameters may entered into the TPS. Dose distributions are subsequently calculated and compared to the original Monte Carlo-derived dose distributions. The planning technique of the present invention reproduces brachytherapy dose distributions for the various applicator types and produces dosimetric agreement within 2% compared to the Monte Carlo-derived dose distributions. Agreement between Monte Carlo-derived and planned dose distributions improve as the spatial resolution of the fitted dosimetry parameters improve. The system and method of the present invention incorporates complex Monte Carlo-based brachytherapy dose distributions into conventional TPS with results that are generalizable to other brachytherapy source types and other TPS.

The present invention provides a system, method, and computer program product for clinical treatment planning of complex Monte Carlo-based brachytherapy dose distributions in clinical situations where the conventional approach is not acceptable and dose distributions present cylindrical symmetry. Using conventional brachytherapy TPS, the system and method of the present invention allows one to replicate dose distributions obtained by Monte Carlo methods for complex multi-source applicators containing high-Z shielding for bounded phantom configurations with a “virtual source” using a modified TG-43 formalism (using either the standard polar or the cylindrical coordinates systems). Clinical implementation of a system and method in accordance with the present invention is described for three brachytherapy treatment modalities where conventional TPS calculations (based on single-source input data) are inadequate.

The present invention includes a system, method, and computer program product for clinical treatment planning of complex Monte Carlo-based brachytherapy dose distributions without the shortcomings of previous techniques. A system, method, and computer program product in accordance with the present invention establishes a new treatment planning technique that may be applied in clinical situations where dose distributions present cylindrical symmetry, but where conventional approaches to treatment planning are not acceptable.

The computer program product is a computer readable storage media for determining a virtual brachytherapy total dose distribution in a patient for radiation therapy treatment planning, and the computer readable storage media includes one or more computer-readable instructions configured to cause one or more computer processors from executing operations including receiving a Monte Carlo dose distribution from a brachytherapy source; identifying a virtual brachytherapy source dose distribution with a cylindrical axis of symmetry; determining an origin location of the virtual brachytherapy source; selecting an active length of a virtual brachytherapy source; deriving a radial dose function along a long axis of the virtual brachytherapy source dose distribution; deriving a 2D anisotropy function of the virtual brachytherapy source; selecting a virtual brachytherapy dose rate constant at a radial distance reference point to reproduce the Monte Carlo dose distribution; and calculating a virtual brachytherapy source dose distribution for a treatment configuration based upon the received Monte Carlo dose distribution.

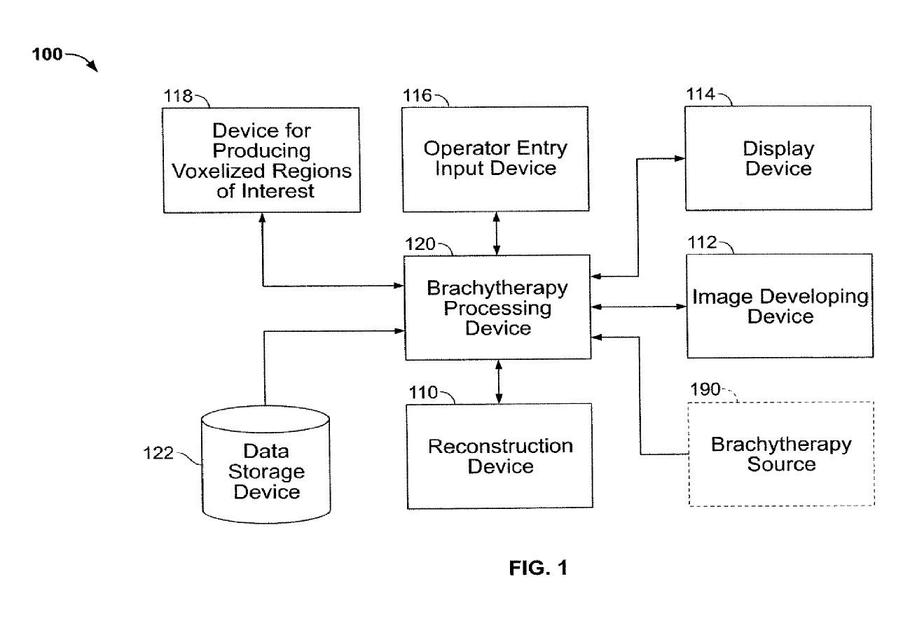

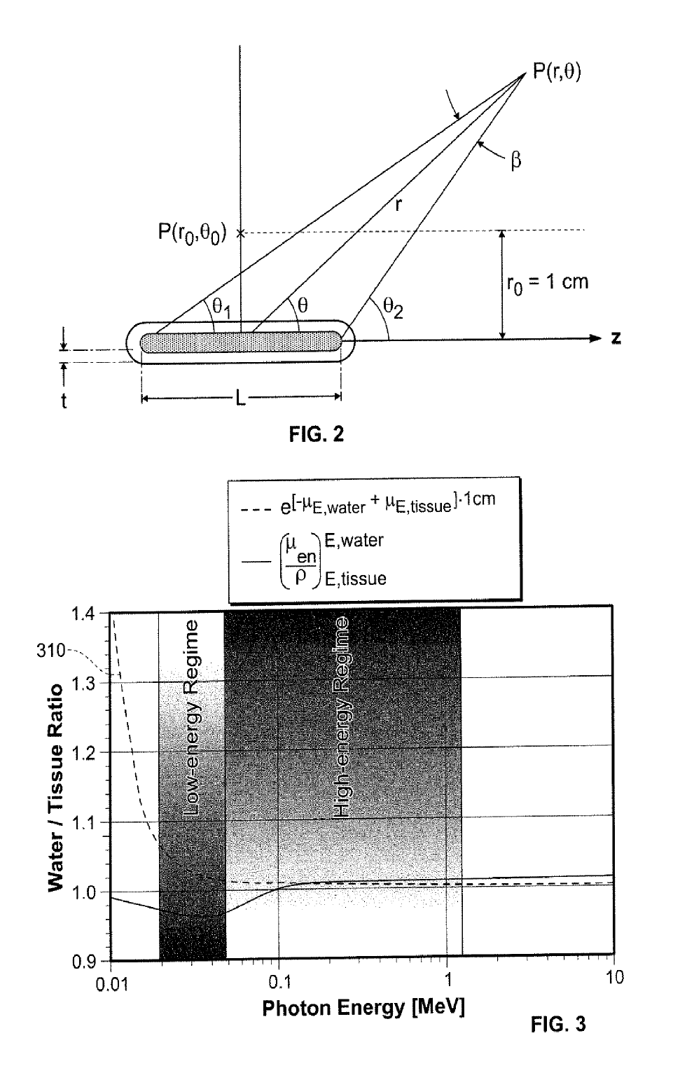

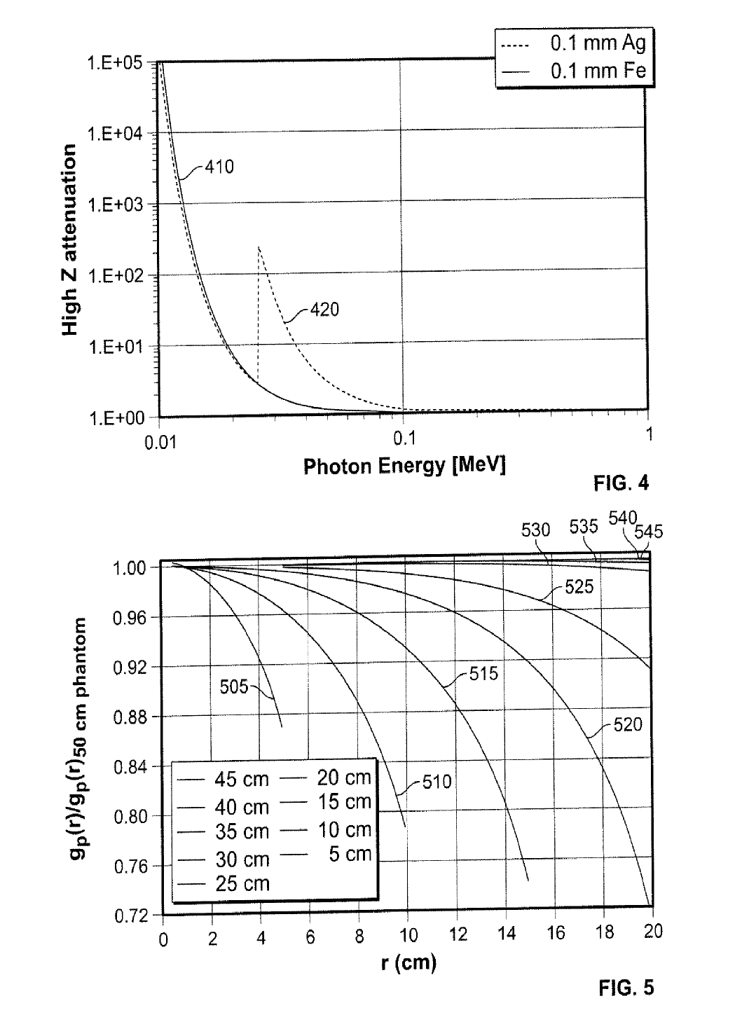

FIG. 1 illustrates an exemplary system of clinical treatment planning of complex, Monte Carlo-based brachytherapy dose distributions in which techniques and methods in accordance with the present invention may be performed. As shown in FIG. 1, clinical treatment planning system 100 includes brachytherapy processing device 120, data storage device 122, operator entry input device 116, device for producing voxelized regions of interest 118, image developing device 112, and display device 114. A user (not shown) may be a physician, physicist, individual, group, organization, client, server, and the like. Users may access clinical treatment planning system 100 performing a method in accordance with the present invention. For clarity and brevity, in FIG. 1 a single clinical treatment planning system 100 is shown, but it should be understood that any number of clinical treatment planning systems may be accessed or connected by a communication network with which to perform methods in accordance with the invention.

The system 100 includes a brachytherapy processing device 120 configured to receive a Monte Carlo dose distribution from a brachytherapy source 190. The brachytherapy processing device 120 is further configured to identify a virtual brachytherapy source dose distribution with a cylindrical axis of symmetry, determine an origin location of the virtual brachytherapy source, and select an active length of a virtual brachytherapy source. Additionally, the brachytherapy processing device 120 is also configured to derive a radial dose function along a long axis of the virtual brachytherapy source dose distribution, derive a 2D anisotropy function of the virtual brachytherapy source, and select a virtual brachytherapy dose rate constant at a radial distance reference point to reproduce the Monte Carlo dose distribution. The brachytherapy processing device 120 is further configured to calculate a virtual brachytherapy source dose distribution for a treatment configuration based upon the received Monte Carlo dose distribution.

The determination of the virtual brachytherapy dose distribution is made in accordance with patient data and configuration data related to the administration of the brachytherapy dose. The device for producing voxelized regions-of-interest 118 for a patient to be treated may use clinical imaging data from multiple modalities, such as computed tomography images, MRI images, and the like from which to produce the voxelized regions-of-interest. These imaging data and voxelized regions-of-interest may be stored on data storage device 122 and retrieved and sent to image reconstruction device 110 to produce and reconstruct cross-sectional patient or other clinical data to be used in a radiation therapy plan. Additionally, the system 100 of the present invention includes an image-developing device 112 for developing a translucent or other image of the patient to be used in visualizing a therapy treatment plan on display device 114. The developed image may be viewed from a predetermined point-of-view or may be adjusted by an operator using operator entry input device 116. The system 100 further includes display device 114 for displaying the received voxelized regions-of-interest and the reconstructed images produced by the radiation therapy planning system 100. The operator entry input device 116 may be further configured for determining the isocenter location of a planned dose and a radiation angle over the received images. The operator entry input device 116 may also be configured to display and adjust a radiation field over the translucent image displayed on the display device 114. Likewise, the operator entry input device 116 may be further configured to adjust, modify, and change the isocenter location, the radiation angle, and the radiation field over the cross-sectional images displayed on the display device 114.

As outlined above, a system and method in accordance with the present invention determines a virtual brachytherapy total dose distribution in a patient for radiation therapy treatment planning. A Monte Carlo dose distribution is received from a brachytherapy source and a virtual brachytherapy source dose distribution with a cylindrical axis of symmetry is identified. An active length of a virtual brachytherapy source is selected and a radial dose function along a long axis of the virtual brachytherapy source dose distribution is derived. The system and method then determines An origin location of the virtual brachytherapy source is determined, and a 2D anisotropy function of the virtual brachytherapy source is derived. The system and method selects a virtual brachytherapy dose rate constant at a radial distance reference point to reproduce the Monte Carlo dose distribution and calculates a virtual brachytherapy source dose distribution for a treatment configuration based upon the received Monte Carlo dose distribution.

Monte Carlo dose distributions for complex configurations are generally not formatted in the geometry of single-source dose distributions for which the TG-43 dosimetry formalism is defined. Thus, the system and method of the present invention converts collimated, cylindrically symmetric dose distributions for complex treatment configurations to the TG-43 formalism as a virtual source. The virtual source may subsequently be applied in the TPS to replicate the MC-derived dose distribution from the applicator.

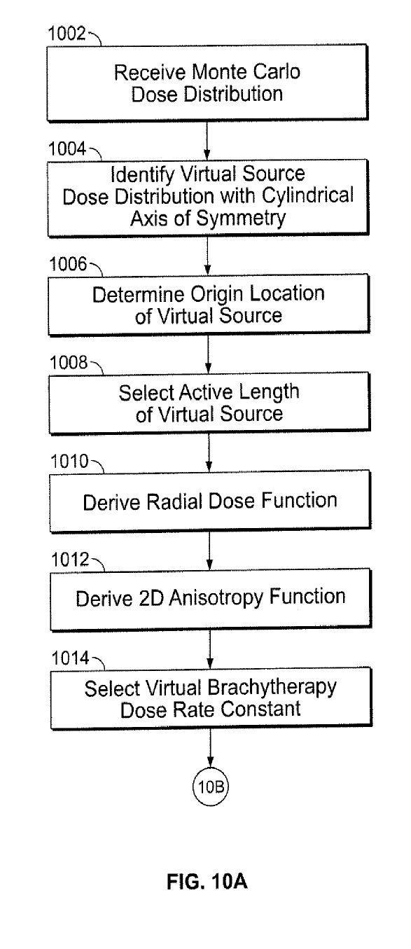

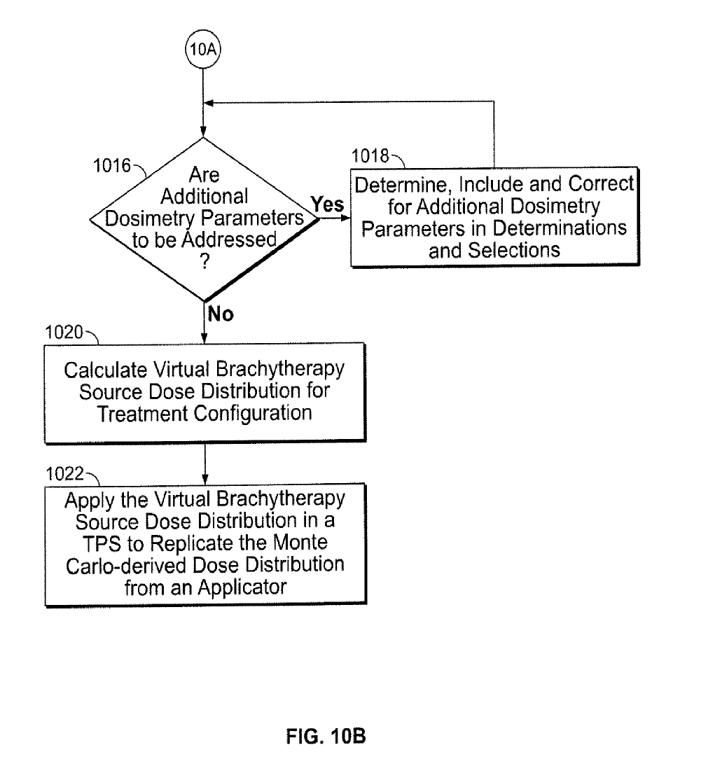

FIGS. 10A-10B are process flow diagrams that illustrate a method of determining a virtual brachytherapy total dose distribution in a patient for radiation therapy treatment planning in accordance with the present invention. The process begins in step 1002 when brachytherapy processing device 120 receives a Monte Carlo dose distribution from a brachytherapy source 190. Brachytherapy source 190 may include Iodine-125, Palladium-103, Cesium-131, Cesium-137, Iridium-192, Cobalt-60, as examples, but may include any high dose rate or low dose rate energy sources. The Monte Carlo dose distribution may be cylindrically symmetric or may be a complex configuration.

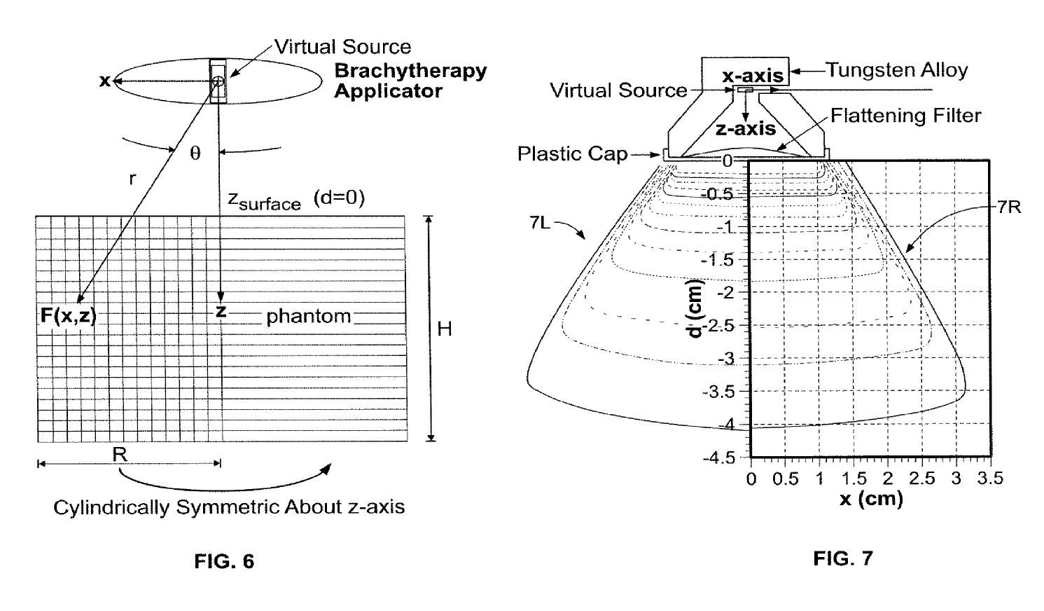

In step 1004, brachytherapy processing device 120 identifies a virtual brachytherapy source dose distribution with a cylindrical axis of symmetry. The method of the present invention obtains TG-43 data for virtual sources by identifying the dose distribution cylindrical axis of symmetry. In the system and method of the present invention, radial dose function g(r) and 2D anisotropy function F(r, θ) are obtained by positioning the coordinate system origin of the virtual source along the dose distribution cylindrical axis of symmetry (i.e., z axis) in step 1006 and as shown schematically in FIG. 6. The 2D anisotropy could be represented as either F(r, θ) or F(x, z). Radial distance r and polar angle θ are defined as for the AAPM TG-43 polar coordinate system. The cylindrical coordinate system includes parameters such as z distance from the virtual source along the z axis and x distance normal to and measured from the z axis

Distance along this axis between the patient surface z_{surface} and coordinate system origin of the virtual source was set to minimize g(r) interpolation errors and roughly approximated the average position of the source(s) used in the applicators. Further, the radial dose function may be derived to account for dose falloff and attenuation along the central longitudinal axis. The depth d into the patient and the distance from the virtual source to z_{surface} are related d=z=z_{surface} except for the eye plaque where d=z−z_{surface}−0.1 cm.

In some brachytherapy applications, the sources are positioned outside the patient at a radial distance greater than the AAPM TG-43 normalization reference point (r₀=1 cm). The system and method of the present invention accounts for these applications as discussed below with regard to the skin and breast applicators. For the conventional TG-43 dose calculation formalism, radial dose function g(r) is normalized at this reference point.

In step 1008, the virtual source length L is selected with a very small value, such as L=0.01 cm, in order to define the virtual source as linear and to include a 2D anisotropy function F(r, θ) in the calculation of dose distributions. Further, the linear virtual source length may be approximated as a point. The system and method of the present invention uses the 2D brachytherapy dosimetry formalism with a point-source geometry function to minimize g(r) interpolation errors while using F(r, θ) instead of φₐₙ(r).

The applicators are oriented with a central axis into the patient along θ=0° as shown in FIG. 6. Returning to FIG. 10, in step 1010, radial dose function g(r) is derived to account for dose falloff and attenuation along the central axis, that is the z axis. After calculating g(r) along the longitudinal axis of the virtual source using:

$$g ( r ) = \frac{\dot{D} ( r , θ = 0 ° )}{\dot{D} ( {r}_{0} θ = 0 ° )} {( \frac{r}{{r}_{0}} )}^{2} ,$$

a 2D anisotropy function F(r, θ) is selected in step 1012 to minimize differences with the original MC data. Although g(r) was derived along θ=0°, it was introduced in the therapy planning system as if it were derived along the standard profile of θ₀=90°. Because the dose profile used to obtain g(r) in the method of the present invention was along θ=0°, F(r,θ=0°) was set to unity for dose profile reproduction along the axis of symmetry, and F(r,θ₀=90°) was also set to unity for consistency with the TG-43 formalism. Additionally, dose derived along the x axis (θ₀=90°) was located outside the patient and is not of clinical relevance.

In step 1014 a value for the dose rate constant A is selected in order to reproduce the absolute Monte Carlo-calculated dose value data using the pointlike geometry factor and the derived g(r) and F(r, θ) values. The virtual brachytherapy dose rate constant A may be selected at a radial distance reference point to reproduce the Monte Carlo dose distribution. The virtual brachytherapy dose rate constant A may use the approximated point linear virtual source and the derived 2D anisotropy function. However, the radial distance reference point may be different from the AAPM TG-43 normalization reference point. The radial distance reference point may be chosen to minimize radial dose function interpolation errors.

In step 1016, if there are additional dosimetry parameters present in the treatment configuration that need to be addressed in the calculation, the method of the present invention progresses to step 1018. The additional dosimetry parameters may include patient scatter conditions, material heterogeneities between the patient and the virtual brachytherapy source, non-water radiation attenuation, dose attenuation in collimated regions of the virtual brachytherapy source dose distribution, high-Z shielding, and the like. If such parameters are to be included in the determination of the virtual brachytherapy total dose distribution, these treatment configuration parameters are included in the determination steps and selections in step 1018.

Once the treatment configuration is finalized, the virtual brachytherapy source dose distribution is calculated in step 1020 for the treatment configuration based upon the received Monte Carlo dose distribution. The virtual brachytherapy source dose distribution may comply with the TG-43 dosimetry formalism. The resulting virtual brachytherapy source dose distribution may then be applied in a therapy planning system (TPS) to replicate the Monte Carlo-derived dose distribution from an applicator.

### Brachytherapy Applicator Examples, Dose Calculations, and Comparisons

To illustrate the effectiveness of the system and method of the present invention, a number of brachytherapy devices were evaluated. For example, skin applicators, breast applicators, and ocular melanoma eye plaques were used to demonstrate the advantages of a system and method of the present invention. Except very close to the eye plaque or due to HDR ¹⁹²Ir source polar angle anisotropy for the skin applicators, dose distributions were readily approximated in the treatment configurations as cylindrically symmetric.

Dosimetry parameters deduced using the method of the present invention were entered into a therapy planning system (TPS). For calculation of 2D dose distributions, the TPS permits 2D parameter entry of polar coordinate system F(r, θ) data or cylindrical coordinate system F(x, z) data. While brachytherapy dose calculations may use either cylindrical and polar coordinate systems, in the examples below, the system and method of the present invention employs a cylindrical coordinate system in order to match the format of the MC-based results for the three applicator types examined. Values for the virtual brachytherapy dose rate constant A were chosen to correlate the TPS results with simulated absolute dose rates. Thus, the MC-derived dose distribution for a complex arrangement of materials and radiation sources was reduced to one virtual source in an FDA-approved conventional TPS.

Skin Applicators

The skin applicators used in an exemplary system of the present invention are cone-shaped applicators made of tungsten alloy designed to treat skin tumors. The skin applicators are 2 cm and 3 cm in diameter and are referred to as model 2 and model 3, respectively. In FIG. 7, a schematic view of the model 2 applicator is shown together with its corresponding MC-based isodose curves. FIG. 7 shows a dose distribution comparison of Monte Carlo input and TPS output using the method of the present invention for model 2 (2 cm diameter) skin applicator. TPS isodose distributions are shown on the left side 7L while MC-based isodose curves are on the right 7R. From the bottom curves, the isodose lines are 10%, 15%, 20%, 30%, 40%, 50%, 60%, 70%, 80%, 90%, 100%, 110%, and 120%. The HDR ¹⁹²Ir source is located 1.6 cm from the applicator outside the surface and moved in parallel to the skin surface. A tungsten-alloy aperture collimates the ¹⁹²Ir emissions, and a tungsten-alloy flattening filter makes uniform the dose profiles at a given depth. Dose distributions were calculated every 0.05 cm in depth and radial extent for a right cylinder of radius R=5 cm and height H=5 cm. Since the most superficial voxel was bounded by xz planes from 0≦d≦0.050 cm, dose calculated in this voxel was assigned an average depth of 0.025 cm. Voxel depths increased sequentially by 0.050 cm. With dose simulated in this cylindrical coordinate system, the centrally positioned voxel was bound by radii of 0-0.050 cm, increasing sequentially by 0.050 cm. The cylindrical radii were assigned positions of 0.025, 0.075, 0.125 cm, and so on. For the conventional planning approach, a single HDR ¹⁹²Ir source was positioned 1.6 cm distant and in parallel to the patient surface.

For these skin applicators, the radial dose function g(r) data entered into the TPS were in the range 1.0≦r≦8.4 cm with 0.2 cm increments. Due to the solid angle of a single HDR ¹⁹²Ir source, a 1/r² geometry function fitted the dose falloff and permitted 0.2 cm g(r) increments. The cylindrical coordinate system F(x, z) data were in the ranges 0.025≦x≦4.975 cm and 1.625≦z≦6.575 cm, both with 0.05 cm increments. Thus, 38 and 10,000 data points were used for g(r) and F(x, z) characterizations, respectively. From these data, the virtual source origin for both applicators (i.e., model 2 and model 3) was set to z_{surface}=1.6 cm.

Breast Applicators

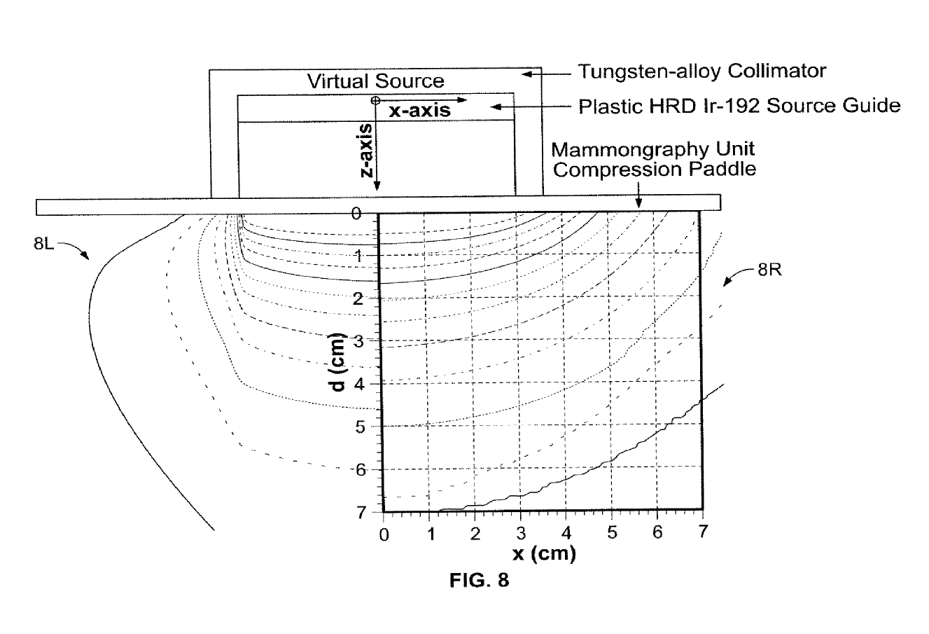

The breast applicators in an exemplary system of the present invention may administer radiation from an HDR ¹⁹²Ir brachytherapy source in a noninvasive manner for peripheral treatment of breast cancer. The applicators are made of tungsten alloy and are shaped as an open right cylinder as shown in FIG. 8. A catheter to direct an HDR ¹⁹²Ir source follows the inside circular edge forming a ring of dwell positions in order to collimate ¹⁹²Ir photons and apply dose uniformly (dwell step of 10 mm circumferentially). The distance from patient skin surface to circular ring plane for HDR ¹⁹²Ir dwells is 2.675 cm. As with the skin applicators, these breast applicators come in different diameters, models B4, B5, B6, B7, and B8 for 4, 5, 6, 7, and 8 cm diameter applicators, respectively. The breast applicators are designed for treatment of deep-seated lesions using opposing beams and are compatible with available HDR ¹⁹²Ir systems. The position of the applicators is determined using mammographic image guidance which is integrated into the treatment configuration platform. Dose distributions for the breast applicators may be determined using a number of Monte Carlo codes. Voxels are oriented with cylindrical geometry spaced every 0.1 cm in a right cylindrical breast phantom with R=15 cm and H=8 cm.

The radial dose function g(r) data entered into the TPS for the breast applicators were in the range 1≦r≦18.4 cm with 0.1 cm increments. Accounting for the 0.275 cm thick compression paddle, z_{surface}=2.675 cm. The cylindrical coordinate system F(x, z) data were in the ranges 0≦x≦15 cm and 2.675≦z≦10.675 cm, both with 0.1 cm increments. Thus, 161 and 12,231 data points were used for g(r) and F(x, z) characterizations, respectively. For the conventional source technique, a ring of HDR ¹⁹²Ir model V2 sources (1 cm circumferential dwell spacing) was positioned at z=0 and in parallel to the phantom surface with z_{surface}=2.675 cm.

FIG. 8 shows a dose distribution comparison of TPS output using the method of the present invention 8L (left) and the conventional planning technique 8R (right) for the 6 cm diameter breast applicator. These results were representative of comparisons for all five round breast applicators, indicating significant differences between the method of the present invention and that of the conventional source superposition principle approach. From the bottom curves, the isodose lines are 10%, 20%, 30%, 40%, 50%, 60%, 70%, 80%, 90%, 100%, 110%, and 120%.

Eye Plaques

The eye plaques in an exemplary system of the present invention contain a silastic seed carrier to accommodate low-energy LDR photon-emitting seeds. A gold-alloy backing may be used to shield radiation not directed to the eye lesion. Different plaque diameters with different seed configurations are available depending on the extent and position of the lesion. Dose distributions in homogeneous and heterogeneous media were obtained for ¹⁰³Pd, ¹²⁵I, or ¹³¹Cs seeds using a Monte Carlo code. Additional MC simulations using the same approach were also performed in this example using depths ranging from −0.1 cm (i.e., outer sclera) to 3.06 cm in 0.02 cm increments for H=3.16 cm and R=1.5 cm in 0.05 cm increments in a 15 cm radius spherical water phantom. Further, the number of histories was increased to 10⁹ histories for ˜0.4% statistical uncertainties at a depth of 0.5 cm.

In the case of the eye plaques, distances from patient eye surface to the virtual source origin (z_{surface}) were set at 0.4, 0.5, and 0.6 cm for ¹⁰³Pd (model 200), ¹²⁵I (model 6711), and ¹³¹CS (model CS-1 Rev2), respectively. This variability was used to optimally smooth the g(r) fits of MC-derived dose falloff. For ¹⁰³Pd, the g(r) data entered into the TPS were in the range 0.3≦r≦3.46 cm with 0.02 cm increments, and the cylindrical coordinate system F(x, z) data were in the ranges 0≦x≦1.5 cm (0.05 cm increments) and 0.30≦z≦3.46 cm (0.02 cm increments). For ¹²⁵I and ¹³¹Cs, g(r) data were in the ranges 0.4≦z≦3.56 cm and 0.5≦z≦3.66 cm, respectively, and had the same spatial resolution and cylindrical range F(x, z). Thus, for each radionuclide examined, 176 and 4,929 data points were used for g(r) and F(x, z) characterizations, respectively.

Once brachytherapy dosimetry parameters were entered into the TPS, treatment plans using these data were performed to compare the original MC data to resultant dose calculations using the system and method of the present invention, as well as to conventional brachytherapy dose calculations based on the standard TG-43 brachytherapy dosimetry formalism. While generally important only for isodose line visualization, the TPS dose calculation grid was set to 0.1 cm to minimize volumetric averaging perturbations. Using the TPS, dose values for the calculation points were independent of dose grid resolution, size, and position. A quantitative comparison was made by positioning dose calculation points throughout the irradiated volume. The location of these points is specified in Table 2 below. Points without dose listed in Table 2 were positioned within the plaque. For the skin applicators, breast applicators, and eye plaque applicators, dose rate was normalized to 100% at depths of 0.3, 1.0, and 0.5 cm along the central axis, respectively. While not directly related to clinical prescriptions, this normalization approach permits simple derivation of differences between the three techniques. Dose rates at these points obtained from the method of the present invention technique, D_{Tufts}, or the conventional TG-43 based approach, D_{TG-43}, were compared to the original MC data D_{MC} as D_{Tufts}/D_{MC} or D_{TG-43}/D_{MC}. Agreement between the planning methods was evaluated using two criteria: 1.00±0.02 (i.e., ±2%) based on AAPM TG-56 recommendations or a more generous criterion of 1.00±0.05 (i.e., ±5%) was used to determine sensitivity of agreement to a threshold value. In Table 2, dose rates are shown at calculation points for the 2 cm skin applicator (model 2), the 6 cm breast applicator (B6), and a 16 mm diameter eye plaque (¹²⁵I). Data were normalized to 100% on the central axis at depths of 0.3, 1.0, and 0.5 cm, respectively. For the eye plaque, blank data entries indicate positions located within the eye plaque. Significantly better agreement to Monte Carlo results was observed when comparing dose rates obtained using the method of the present invention versus those obtained using source superposition of conventional TG-43 dose calculation formalism. Values in boldface indicate dose rate ratios outside ±5% agreement with Monte Carlo, while “NA” indicates r>10 cm where HDR ¹⁹²Ir g(r) data were not entered into the TPS.

Analysis of Results of Dose Calculations and Comparisons

As shown, in Table 2, the method of the present invention presented significantly better agreement to Monte Carlo results compared to the dose rates obtained using the source superposition techniques of conventional TG-43 dose calculation formalism.

Though the source input and dose calculation grid sizes were much smaller than typically used for external beam treatment planning, the time needed to calculate dose was less than 1 s in all cases since the complex dose distribution for one virtual source was precalculated during the virtual source commissioning phase and data were stored in the TPS.

TABLE 2

| Skin app. | Monte Carlo (GEANT4) | | | | | | |
|---|---|---|---|---|---|---|---|
| Model 2 | lateral distance (cm) | | | | | | |
| Depth (cm) | 0.025 | 0.525 | 1.025 | 1.525 | 2.025 | 2.525 | 3.025 |
| 0.025 | 137.4 | 136.9 | 133.8 | 8.41 | 5.52 | 4.37 | 3.76 |
| 0.300 | 100.0 | 99.40 | 99.90 | 12.91 | 5.10 | 3.94 | 3.23 |
| 05.25 | 79.30 | 80.93 | 80.75 | 29.13 | 5.22 | 3.79 | 3.06 |
| 1.025 | 51.30 | 52.13 | 51.28 | 49.55 | 9.79 | 3.67 | 2.78 |
| 1.525 | 35.80 | 36.10 | 35.45 | 34.68 | 28.65 | 5.32 | 2.74 |
| 2.025 | 26.10 | 26.35 | 25.90 | 25.30 | 24.28 | 12.76 | 3.50 |
| 2.525 | 19.84 | 19.95 | 19.66 | 19.15 | 18.60 | 16.96 | 6.16 |
| 3.025 | 15.58 | 15.60 | 15.37 | 15.00 | 14.57 | 13.96 | 10.89 |
| Skin app. | Method of Present Invention (PINNACLE) | | | | | | |
| Model 2 | lateral distance (cm) | | | | | | |
| Depth (cm) | 0.025 | 0.525 | 1.025 | 1.525 | 2.025 | 2.525 | 3.025 |
| 0.025 | 136.2 | 135.4 | 130.4 | 8.29 | 5.50 | 4.36 | 3.75 |
| 0.300 | 100.0 | 99.24 | 99.72 | 13.15 | 5.19 | 3.99 | 3.28 |
| 05.25 | 79.20 | 78.75 | 78.75 | 29.13 | 5.23 | 3.78 | 3.05 |
| 1.025 | 51.35 | 51.14 | 50.23 | 48.51 | 9.90 | 3.70 | 2.79 |
| 1.525 | 35.93 | 35.64 | 34.96 | 34.15 | 28.40 | 5.39 | 2.76 |
| 2.025 | 26.30 | 26.14 | 25.68 | 25.05 | 24.02 | 12.85 | 3.54 |
| 2.525 | 19.98 | 19.85 | 19.54 | 19.04 | 18.47 | 16.86 | 6.25 |
| 3.025 | 15.68 | 15.52 | 15.33 | 14.94 | 14.51 | 13.91 | 10.89 |

| Skin app. | TG-43 (PINNACLE) | | | | | | |
|---|---|---|---|---|---|---|---|
| Model 2 | lateral distance (cm) | | | | | | |
| Depth (cm) | 0.025 | 0.525 | 1.025 | 1.525 | 2.025 | 2.525 | 3.025 |
| 0.025 | 134.9 | 122.3 | 95.94 | 70.61 | 51.22 | 37.43 | 28.03 |
| 0.300 | 100.0 | 92.90 | 76.88 | 59.78 | 45.50 | 34.45 | 26.41 |
| 05.25 | 79.86 | 75.20 | 64.43 | 52.01 | 40.94 | 31.84 | 24.92 |
| 1.025 | 52.64 | 50.55 | 45.51 | 38.94 | 32.94 | 26.49 | 21.51 |
| 1.525 | 37.26 | 36.16 | 33.46 | 29.77 | 25.76 | 21.95 | 18.43 |
| 2.025 | 27.72 | 27.08 | 25.47 | 23.26 | 20.73 | 18.18 | 15.78 |
| 2.525 | 21.37 | 20.99 | 19.95 | 18.54 | 16.89 | 15.15 | 13.44 |
| 3.025 | 16.95 | 16.71 | 16.00 | 15.04 | 13.93 | 12.73 | 11.49 |

| Breast app. | Monte Carlo (MCNP5) | | | | | | | |
|---|---|---|---|---|---|---|---|---|
| B-6 | lateral distance (cm) | | | | | | | |
| Depth (cm) | 0.0 | 1.0 | 2.0 | 3.0 | 4.0 | 5.0 | 6.0 | 7.0 |
| 0.0 | 145.1 | 145.6 | 143.3 | 123.5 | 12.80 | 8.60 | 7.34 | 6.02 |
| 1.0 | 100.0 | 98.94 | 94.33 | 82.93 | 30.00 | 16.15 | 9.03 | 5.17 |
| 2.0 | 70.06 | 68.50 | 64.70 | 57.11 | 27.93 | 17.21 | 11.51 | 7.96 |
| 3.0 | 49.92 | 48.88 | 46.10 | 41.12 | 23.64 | 15.76 | 11.16 | 8.17 |
| 4.0 | 36.20 | 35.76 | 33.85 | 30.53 | 19.49 | 13.75 | 10.13 | 7.68 |
| 5.0 | 27.08 | 26.73 | 25.38 | 23.16 | 15.86 | 11.71 | 8.92 | 6.93 |
| 6.0 | 20.74 | 20.31 | 19.38 | 17.83 | 12.89 | 9.82 | 7.68 | 6.10 |
| 7.0 | 15.71 | 15.60 | 14.97 | 13.85 | 10.43 | 8.14 | 6.50 | 5.26 |
| Breast app. | Method of Present Invention (PINNACLE) | | | | | | | |
| B-6 | lateral distance (cm) | | | | | | | |
| Depth (cm) | 0.0 | 1.0 | 2.0 | 3.0 | 4.0 | 5.0 | 6.0 | 7.0 |
| 0.0 | 145.4 | 145.1 | 143.6 | 124.1 | 12.81 | 8.60 | 7.35 | 6.03 |
| 1.0 | 100.0 | 99.14 | 94.70 | 83.21 | 30.01 | 16.17 | 9.04 | 5.19 |
| 2.0 | 70.36 | 68.78 | 64.92 | 57.21 | 27.93 | 17.25 | 11.55 | 7.97 |
| 3.0 | 50.05 | 49.00 | 46.11 | 41.15 | 23.70 | 15.81 | 11.19 | 8.17 |
| 4.0 | 36.21 | 35.81 | 33.89 | 30.62 | 19.56 | 13.79 | 10.15 | 7.69 |
| 5.0 | 27.14 | 26.80 | 25.46 | 23.24 | 15.89 | 11.72 | 8.93 | 6.94 |
| 6.0 | 20.79 | 20.37 | 19.42 | 17.86 | 12.91 | 9.83 | 7.70 | 6.12 |
| 7.0 | 15.71 | 15.62 | 14.99 | 13.87 | 10.47 | 8.17 | 6.52 | 5.27 |
| Breast app. | TG-43 (PINNACLE) | | | | | | | |
| B-6 | lateral distance (cm) | | | | | | | |
| Depth (cm) | 0.0 | 1.0 | 2.0 | 3.0 | 4.0 | 5.0 | 6.0 | 7.0 |
| 0.0 | 141.7 | 141.5 | 138.5 | 125.0 | 99.66 | 73.77 | 53.99 | 37.69 |
| 1.0 | 100.0 | 98.42 | 93.50 | 84.19 | 70.95 | 56.55 | 44.09 | 32.18 |
| 2.0 | 71.67 | 70.37 | 66.46 | 60.45 | 52.60 | 44.07 | 35.98 | 22.80 |
| 3.0 | 52.70 | 51.80 | 49.09 | 45.11 | 40.16 | 34.73 | 27.22 | 18.83 |
| 4.0 | 39.70 | 39.10 | 37.26 | 34.58 | 31.31 | 25.45 | 18.05 | 15.48 |
| 5.0 | 30.48 | 30.09 | 28.85 | 27.01 | 22.54 | 16.24 | 14.52 | 8.86 |
| 6.0 | 23.84 | 23.57 | 22.71 | 18.43 | 13.98 | 8.67 | 7.97 | 6.98 |
| 7.0 | 18.90 | 12.07 | 7.74 | 7.49 | 7.26 | 2.74 | 2.33 | NA |

| Eye plaque | Monte Carlo (MCNP5) | | | | | | |
|---|---|---|---|---|---|---|---|
| 125₁ | lateral distance (cm) | | | | | | |
| Depth (cm) | 0.0 | 0.2 | 0.4 | 0.6 | 0.8 | 1.0 | 1.2 |
| −0.0 | 364.9 | 399.5 | | | | | |
| 0.0 | 299.7 | 304.1 | 325.0 | 410.5 | | | |
| 0.1 | 239.5 | 240.8 | 244.8 | 225.7 | | | |
| 0.3 | 153.4 | 152.2 | 144.6 | 121.8 | 82.4 | 13.5 | 4.7 |
| 0.5 | 100.0 | 98.7 | 91.8 | 77.9 | 59.0 | 35.6 | 14.6 |
| 1.0 | 39.4 | 38.9 | 36.7 | 32.9 | 28.1 | 23.0 | 18.1 |
| 1.5 | 18.6 | 18.5 | 17.8 | 16.6 | 14.9 | 13.1 | 11.2 |
| 2.0 | 10.0 | 9.9 | 9.7 | 9.2 | 8.6 | 7.8 | 7.0 |
| Eye plaque | Method of Present Invention (PINNACLE) | | | | | | |
| 125₁ | lateral distance (cm) | | | | | | |
| Depth (cm) | 0.0 | 0.2 | 0.4 | 0.6 | 0.8 | 1.0 | 1.2 |
| −0.0 | 367.0 | 398.8 | | | | | |
| 0.0 | 299.8 | 303.8 | 324.5 | 409.8 | | | |
| 0.1 | 239.6 | 240.5 | 244.5 | 225.4 | | | |
| 0.3 | 153.0 | 152.0 | 144.5 | 121.7 | 82.3 | 13.5 | 4.7 |
| 0.5 | 100.0 | 98.6 | 91.7 | 77.8 | 58.9 | 35.5 | 14.6 |
| 1.0 | 39.4 | 38.8 | 36.6 | 32.8 | 28.1 | 22.9 | 18.0 |
| 1.5 | 18.6 | 18.5 | 17.7 | 16.5 | 14.9 | 13.1 | 11.2 |
| 2.0 | 10.0 | 9.9 | 9.7 | 9.2 | 8.5 | 7.8 | 7.0 |

| Eye plaque | TG-43 (PINNACLE) | | | | | | |
|---|---|---|---|---|---|---|---|
| 12₁5 | lateral distance (cm) | | | | | | |
| Depth (cm) | 0.0 | 0.2 | 0.4 | 0.6 | 0.8 | 1.0 | 1.2 |
| −0.0 | 403.9 | 362.3 | | | | | |
| 0.0 | 309.7 | 299.1 | 385.7 | 622.5 | | | |
| 0.1 | 240.8 | 238.2 | 257.4 | 275.5 | | | |
| 0.3 | 152.4 | 150.6 | 143.9 | 126.2 | 92.8 | 60.8 | 40.3 |
| 0.5 | 100.0 | 98.3 | 91.4 | 79.1 | 62.8 | 46.8 | 34.1 |
| 1.0 | 40.1 | 40.1 | 37.3 | 33.8 | 29.4 | 24.8 | 20.6 |
| 1.5 | 19.3 | 19.1 | 18.4 | 17.2 | 15.8 | 14.1 | 12.4 |
| 2.0 | 10.5 | 10.5 | 10.2 | 9.7 | 9.1 | 8.4 | 7.7 |

Skin Applicators

The original MC-derived dose data and the doses determined using a system and method in accordance with the present invention are shown in FIG. 7 for the model 2 applicator. For the 56 points used for comparison (see Table 2), the maximum and minimum D_{Tufts}/D_{MC} ratios were 1.019 and 0.971 for the model 2 applicator and 1.049 and 0.971 for the model 3 applicator. Differences were attributed to grid coarseness in the gradient region since the largest differences occurred in the high-gradient penumbral region for the model 3 applicator. While 13 points (or 12%) of the 112 points in total were outside the ±2% AAPM TG-56 criterion for agreement, none were outside ±5%.

The conventional planning approach did not account for material heterogeneities nor the significant attenuation of dose within the collimated regions. Material heterogeneities include air in the applicator, the flattening filter that significantly changed the dose profile and dose rate within the collimated aperture, or the plastic cover to absorb electrons. The dose attenuation was in the tungsten-alloy collimated regions. For the 56 points used to compare dose, the maximum and minimum D_{TG-43}/D_{MC} ratios were 9.3 and 0.7 for the model 2 and 9.0 and 0.5 for the model 3. The largest differences occurred in the high-gradient penumbral region for the applicators. Of the 112 sampling points in total, 98 (or 88%) were outside the ±2% AAPM TG-56 criterion for agreement, and 83 points (or 74%) were outside ±5%. Thus, the conventional approach did not accurately predict the MC-based dose distributions in contrast to the method in accordance with the present invention.

Breast Applicators

For the 64 points used to compare dose, the maximum and minimum D_{Tufts}/D_{MC} ratios over the 320 sampling points for all five applicators were 1.004 and 0.995, respectively. Differences were attributed to MC statistical uncertainties (typically ˜0.1%) and g(r) curve fitting. Because the dose obtained using the method in accordance with the present invention for the 320 sampling points for all five applicators were within ±0.5%, the ±2% AAPM TG-56 criterion was easily achieved.

Using the conventional planning approach with rings of HDR ¹⁹²Ir sources positioned above the sampling geometry, the maximum and minimum D_{TG-43}/D_{MC} ratios were 10 and 0.3 for all five breast applicators. This means that the conventional treatment planning approach sometimes either over- or underestimated the dose rate by a factor of 10 or 3, respectively. Of the 320 sampling points in total, 297 (or 93%) were outside the ±2% AAPM TG-56 criterion for agreement, and 257 points (or 80%) were outside ±5%. Again, as shown in FIG. 8, the conventional planning approach did not accurately predict the MC-based dose distributions. The method in accordance with the present invention accounted for the clinical environment with dose to breast tissue while the conventional TPS algorithm produced dose to water results.

Eye Plaques

Of the 45 points used to compare dose in the eye plaque applicators, the maximum and minimum D_{Tufts}/D_{MC} ratios were 1.012 and 0.994 for ¹⁰³Pd, respectively. Differences were attributed to MC statistical uncertainties (typically ˜0.4%) and g(r) curve fitting. For ¹²⁵I and ¹³¹Cs, these ratios were 1.006 and 0.995 and 1.006 and 0.993, respectively. Thus, all sampling points were within the ±2% criterion.

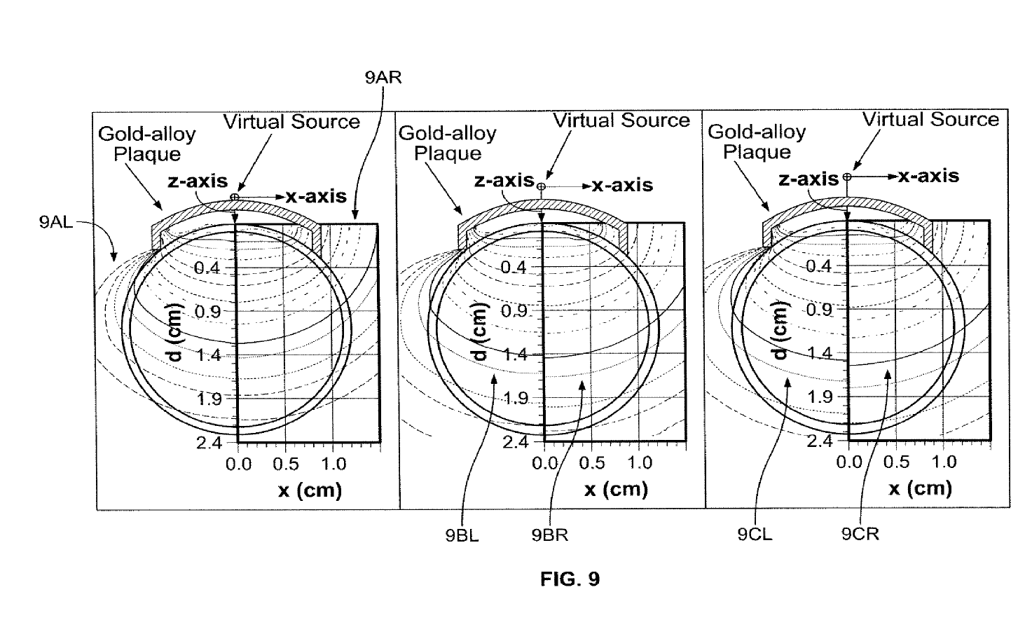

As shown in FIGS. 9A-9C, dose distribution comparisons of the conventional TPS approach and a method in accordance with the present invention were made for a fully loaded 16 mm diameter eye plaque. The left image FIG. 9A, middle image FIG. 9B, and right images FIG. 9C are for ¹⁰³Pd, ¹²⁵I, and ¹³¹I seeds, respectively. In each image, the left-hand side 9AL, 9BL, 9CL is the result of the method of the present invention, and the right-hand side 9AR, 9BR, 9CR is the result of the conventional TG-43 approach. The z axis points from the virtual source to the outer sclera (d=−0.1 cm). From the bottom curves, the isodose lines are 5%, 7.5%, 10%, 15%, 20%, 30%, 40%, 50%, 75%, 100%, 150%, 200%, 250%, 300%, and 350%.

With the conventional TG-43 approach, the maximum and minimum D_{TG-43}/D_{MC} ratios of TG-43 derived dose to Monte Carlo dose were 12 and 0.99 for ¹⁰³Pd, respectively. For ¹²⁵I, these ratios were 8.6 and 0.91. For ¹³¹Cs, these ratios were 8.3 and 0.90. Of the 45 sampling points per radionuclide type, 39 points (or 87%) for ¹⁰³Pd, 33 points (or 73%) for ¹²⁵I, and 35 points (or 78%) for ¹³¹Cs were outside the ±2% AAPM TG-56 criterion for agreement. Even with the more generous ±5% agreement criterion, 30 points (or 67%), 24 points (or 53%), and 28 points (or 62%) were in disagreement for ¹⁰³Pd, ¹²⁵I, and ¹³¹Cs, respectively. Thus, as shown in FIGS. 9A-9C, the conventional treatment planning approach did not accurately predict the MC dose distributions while the method in accordance with the present invention accurately predicted the dose distributions.

While the system and method of the present invention can be extended to other treatment configurations and source configurations, such as for the cylindrical dose distribution of an HDR ¹⁹²Ir vaginal cylinder or elongated brachytherapy sources, the above examples focused on demonstrating the method in three brachytherapy applicator systems. The three applicators used in the example systems were chosen particularly due to limitations of conventional brachytherapy treatment planning methods to accurately depict dose distributions. Once a data set is considered as a reference for clinical dosimetry, such as the MC tabulated values, the goal is to reproduce it as closely as possible with the therapy planning systems (TPSs). A distinction of the method of the present invention is that extrapolation is not used. This conservative approach is especially beneficial in regions where the conventional TG-43 based extrapolations may not apply. Agreement with MC results using the system and method of the present invention was best along the axes of dose symmetry and in areas of uniform dose and was less in agreement near the applicator edge in regions of high-dose gradients. Further, agreement between MC and planned dose distributions improved as the spatial resolution of the fitted dosimetry parameters improved. While the largest errors occurred in the highest dose gradient regions, overall errors reduced from a factor of 10 to just a few percent.

For example, the dose distributions for the eye plaques were initially taken from a data set having about 1400 data points in total but were re-simulated to express over 5000 data points to improve spatial resolution in the high-dose gradient region. This additional step resulted in agreement between the method of the present invention and of the original MC-derived dose improving from about 4% disparities to about 1% disparities. Even with 3D orientation and positioning of the three different seed types, dose calculations using the conventional source superposition principle are limited and could not account for material heterogeneities such as the silastic seed holder nor the significant attenuation of dose within the gold-alloy collimated regions. Again, dose to non-water tissues could be simulated and clinically implemented with the system and method of the present invention. The MC-derived dose distributions were averaged over a given radial distance around the central axis. This radial volume averaging may lead to differences at specific points near the eye plaques within the calculation volume. By design, the skin and breast applicators are cylindrically symmetric. Similarly, the polygon shapes used in eye plaque seed holders were carefully chosen to ensure cylindrical dose symmetry along the central/prescriptive axis. As the distance from the eye plaque decreased or as the plaque size decreases, local dose variations attributed to individual seeds will dominate the overall dose distribution at close distances. For the 16 mm eye plaque example, based on comparing rectilinear mesh data with cylindrically symmetric data at x=0.5 cm, volume averaging may be less than 2% at a depth of 0.3 cm, ˜10% at the inner sclera (0.0 cm), and possibly higher immediately beneath a seed (d<0 cm).

The system and method and computer program product of the present invention was developed to implement complex MC-based brachytherapy dose distributions using conventional TPS. Typical agreement of the dose calculation results between the system and method of the present invention and the Monte Carlo results was within 1% for all three brachytherapy applicator types examined. These results may be generalized to other cylindrically symmetric brachytherapy sources and configurations or to other TPS. Further, in contrast to the conventional TPS algorithm, the system and method of the present invention may be applied to calculate dose to a specific tissue type (e.g., lung, bone, and prostate gland) instead of dose to liquid water as used in the TG-43 formalism. Until advanced brachytherapy dosimetry algorithms become widespread and readily available, the system and method of the present invention may allow users of conventional brachytherapy planning systems that utilize the AAPM TG-43 2D formalism to clinically implement MC-based characterizations of complex, cylindrically symmetric brachytherapy implants.

The present invention presents a significant advancement over previous dose calculation methods by clinically implementing Monte Carlo-based characterizations of complex, cylindrically symmetric brachytherapy implants in conventional brachytherapy planning systems that utilize the AAPM TG-43 2D formalism.

The foregoing description of the present invention provides illustration and description, but is not intended to be exhaustive or to limit the invention to the precise one disclosed. Modifications and variations are possible consistent with the above teachings or may be acquired from practice of the invention. As such, the scope of the invention is defined by the claims and their equivalents.

1. A computer-implemented method of determining a virtual brachytherapy total dose distribution in a patient for radiation therapy treatment planning, the method comprising:
receiving, by a brachytherapy processing device, a Monte Carlo dose distribution from a brachytherapy source;
identifying, by the brachytherapy processing device, a virtual brachytherapy source dose distribution with a cylindrical axis of symmetry;
determining, by the brachytherapy processing device, an origin location of the virtual brachytherapy source;
selecting an active length of a virtual brachytherapy source;
deriving, by the brachytherapy processing device, a radial dose function along a long axis of the virtual brachytherapy source dose distribution;
deriving, by the brachytherapy processing device, a 2D anisotropy function of the virtual brachytherapy source;
selecting a virtual brachytherapy dose rate constant at a radial distance reference point to reproduce the Monte Carlo dose distribution; and
calculating, by the brachytherapy processing device, a virtual brachytherapy source dose distribution for a treatment configuration based upon the received Monte Carlo dose distribution.

2. The computer-implemented method of claim 1, wherein deriving the radial dose function includes positioning a coordinate system along a dose distribution cylindrical axis of symmetry.

3. The computer-implemented method of claim 1, wherein the Monte Carlo dose distribution is cylindrically symmetric.

4. The computer-implemented method of claim 1, wherein the Monte Carlo dose distribution is a complex configuration.

5. The computer-implemented method of claim 1 further comprising:
determining dosimetry parameters including at least one of patient scatter conditions, material heterogeneities, non-water radiation attenuation, and high-Z shielding.

6. The computer-implemented method of claim 5 further comprising:
correcting for at least one of material heterogeneities between the patient and the virtual brachytherapy source, dose attenuation in collimated regions of the virtual brachytherapy source dose distribution, patient scatter conditions, or high-Z shielding.

7. The computer-implemented method of claim 1, wherein the virtual brachytherapy source dose distribution complies with the TG-43 dosimetry formalism.

8. The computer-implemented method of claim 1, wherein the radial dose function is derived by positioning the origin location of the virtual brachytherapy source along the dose distribution cylindrical axis of symmetry.

9. The computer-implemented method of claim 8, wherein the radial dose function is derived to account for dose falloff and attenuation along the central longitudinal axis.

10. The computer-implemented method of claim 1, wherein the 2D anisotropy function is derived by positioning the origin location of the virtual brachytherapy source along the dose distribution cylindrical axis of symmetry.

11. The computer-implemented method of claim 10, wherein the 2D anisotropy function is derived to minimize differences with the Monte Carlo dose distribution from the virtual brachytherapy source.

12. The computer-implemented method of claim 1, wherein the radial distance reference point is not equal to the AAPM TG-43 normalization reference point.

13. The computer-implemented method of claim 12, wherein the radial distance reference point minimizes radial dose function interpolation errors.

14. The computer-implemented method of claim 1, wherein the active length of the virtual brachytherapy source defines the virtual source as linear and includes the 2D anisotropy function in calculating the virtual brachytherapy source dose distribution.

15. The computer-implemented method of claim 14, wherein the linear virtual source length is approximated as a point.

16. The computer-implemented method of claim 15, wherein the virtual brachytherapy dose rate constant reproduces the Monte Carlo dose rate distribution using the approximated point linear virtual source and the derived 2D anisotropy function.

17. The computer-implemented method of claim 1 further comprising:
applying the virtual source dose distribution for the treatment configuration to a therapy planning system to replicate the received Monte Carlo dose distribution from the brachytherapy source.

18. A radiation therapy planning system configured to determine a virtual brachytherapy total dose distribution in a patient for radiation therapy treatment planning, the system comprising:
a brachytherapy processing device configured to
receive a Monte Carlo dose distribution from a brachytherapy source;
identify a virtual brachytherapy source dose distribution with a cylindrical axis of symmetry;
determine an origin location of the virtual brachytherapy source;
select an active length of a virtual brachytherapy source;
derive a radial dose function along a long axis of the virtual brachytherapy source dose distribution;
derive a 2D anisotropy function of the virtual brachytherapy source;
select a virtual brachytherapy dose rate constant at a radial distance reference point to reproduce the Monte Carlo dose distribution; and
calculate a virtual brachytherapy source dose distribution for a treatment configuration based upon the received Monte Carlo dose distribution.

19. A computer readable storage media for determining a virtual brachytherapy dose distribution in a patient for radiation therapy treatment planning, the computer readable storage media comprising one or more computer-readable instructions configured to cause one or more computer processors to execute operations comprising:
receiving a Monte Carlo dose distribution from a brachytherapy source;
identifying a virtual brachytherapy source dose distribution with a cylindrical axis of symmetry;
determining an origin location of the virtual brachytherapy source;
selecting an active length of a virtual brachytherapy source;
deriving a radial dose function along a long axis of the virtual brachytherapy source dose distribution;
deriving a 2D anisotropy function of the virtual brachytherapy source;
selecting a virtual brachytherapy dose rate constant at a radial distance reference point to reproduce the Monte Carlo dose distribution; and
calculating a virtual brachytherapy source dose distribution for a treatment configuration based upon the received Monte Carlo dose distribution.
